(19) United States
(12) Patent Application Publication
(10) Pub. No.: US 2025/0288744 A1
(43) Pub. Date: Sep. 18, 2025

(54) Syringe With Integrated Easy-Open Cap

(71) Applicant: Becton, Dickinson and Company, Franklin Lakes, NJ (US)

(72) Inventors: Srinivas Ramakrishnarao Peddi, Pune (IN); Praveen Nalawade, Bangaluru (IN)

(21) Appl. No.: 18/861,164

(22) PCT Filed: Jun. 15, 2023
(86) PCT No.: PCT/US2023/025353

§ 371 Date: Oct. 28, 2024

(30) Foreign Application Priority Data
Jun. 21, 2022 (IN) 202211035493

(51) Int. Cl. A61M 5/31

(52) U.S. Cl. CPC A61M 5/3134; A61M 2005/3104; A61M 2005/3106; A61M 2202/0007; A61M 2202/04; A61M 2205/0216; A61M 2205/3331

(57) ABSTRACT

A syringe includes a barrel having a proximal end. a distal end including a distal opening. and a sidewall extending between the proximal end and the distal end. The syringe further includes a plunger positioned in the barrel for expelling fluid from the barrel through the distal opening and a cap connected to the distal end of the barrel including a connector defining a flow channel through the cap. The cap is movable relative to the barrel between a first position, where fluid flow through the distal opening of the barrel to the flow channel of the connector is prevented. and a second position, where fluid flow through the distal opening of the barrel to the flow channel of the connector can occur.

### CROSS-REFERENCE TO RELATED APPLICATION

The present application claims priority to Indian patent application Ser. No. 202211035493 entitled “Syringe with Integrated Easy-Open Cap” filed Jun. 21, 2022, the entire disclosure of which is hereby incorporated by reference in its entirety.

### BACKGROUND OF THE INVENTION

### Field of the Invention

The present disclosure relates generally to syringes, such as prefilled syringes, and, in particular, to a syringe having an integrated cap, which can be easily opened by a user, preferably using one hand.

### Description of Related Art

Conventional syringes comprise a barrel having an open proximal end and an opposed distal end. A distal tip, sometimes referred to as a luer, projects from the distal end of the syringe barrel and includes a narrow passage or channel in fluid communication with an interior of the syringe barrel. These conventional syringes are often prefilled with medical fluids, such as flush solutions or therapeutic agents, during manufacture. When prefilled with the medical fluid, the syringe barrel can be sealed to prevent contamination of the medical fluid or loss of the medical fluid prior to use. Seals also prevent practitioners from being needlessly exposed to the medical fluid contained in the syringe barrel, which can be particularly important for toxic or otherwise harmful medications.

Some prefilled syringes have stoppers or closures, often referred to as tip caps, mounted over the distal tip of the syringe barrel for sealing the syringe barrel to prevent leakage and to avoid contamination of the medical fluid contained therein. Prior art tip caps are removed from the distal tip of the syringe barrel by the practitioner shortly before using the syringe by grasping the tip cap and pulling the tip cap axially away from the syringe. When a prefilled syringe is capped with a tip cap, it is especially important that a good seal be maintained. This is usually achieved by tightly affixing the tip cap to the distal tip of the syringe barrel. However, when overly tightened, the tip cap can be difficult to remove or may be damaged. Further, a prefilled syringe may be autoclaved after filling and capping so that the prefilled syringe is contained within sterile packaging. The autoclaving procedure can cause interactions between the tip cap and the syringe barrel, which can further increase the difficulty in removing the tip cap from the distal tip of the syringe barrel.

Due to the tight connection between the tip cap and distal tip of the syringe barrel, the practitioner may be required to apply a substantial gripping force to remove the tip cap from the distal end of the syringe barrel. Tightly grasping the tip cap can be difficult for some practitioners who may lack physical strength and dexterity needed to correctly grip the tip cap, especially considering the small size of the typical tip cap. Further, when tightly grasping the tip cap, the practitioner's fingers are in close proximity to the distal tip of the syringe barrel. Accordingly, there is a risk that the practitioner will contact the distal tip of the barrel while removing the tip cap, which can contaminate the distal tip of the syringe barrel.

Once the tip cap is removed, which occurs shortly before a procedure is performed, the luer tip at one end of the syringe barrel is exposed to a non-sterile environment, which provides additional opportunities for undesirable contamination. Further, in some cases, practitioners may remove the tip cap to perform a preparatory action (e.g., to flush air from the syringe barrel) and then reattach the same tip cap to the syringe distal tip prior to performing a fluid delivery procedure. This reattachment of the tip cap is yet another opportunity for contamination of the distal tip of the syringe barrel.

Prefilled syringes are often connected to and used to deliver fluid to a patient through vascular access devices (VADs), such as intravenous (IV) catheters, such as peripheral catheters or central venous catheters. If not properly maintained or if exposed to non-sterile environments, which, as described previously, can occur as the practitioner manipulates or removes the tip cap from the syringe barrel, the VADs can become contaminated, sealed with blood clots, and/or can spread infection. Many medical facilities implement sterile practices and protocols to avoid contamination and to ensure that VADs are used properly and do not become sealed or infected. These protocols often include sterilizing medical devices including the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition. These flush procedures are intended to confirm catheter patency, avoid drug incompatibilities, ensure that the complete drug dose is administered to the patient, prevent thrombus formation, and minimize a risk of bloodstream infections caused by contamination of the VAD.

While sterilization procedures implemented by medical facilities certainly address some contamination risks, improved designs for medical devices that reduce opportunities for contamination and simplify device use are needed. In particular, syringe designs that ensure that the distal tip of the syringe barrel remains sterile and free from contamination throughout a fluid delivery procedure using the VAD will address many current contamination problems. Further, syringe and cap designs that are easy to open or remove and which can be manipulated without touching the syringe distal tip will reduce common contamination risks for conventional prefilled syringes. The syringes and integrated caps design of the present disclosure addresses these issues.

### SUMMARY OF THE INVENTION

According to an aspect of the disclosure, a syringe includes a barrel having a proximal end, a distal end including a distal opening, and a sidewall extending between the proximal end and the distal end. The syringe further includes a plunger positioned in the barrel for expelling fluid from the barrel through the distal opening; and a cap connected to the distal end of the barrel with a connector defining a flow channel through the cap. The cap is movable relative to the barrel between a first position, where fluid flow through the distal opening of the barrel to the flow channel of the connector is prevented, and a second position, where fluid flow through the distal opening of the barrel to the flow channel of the connector can occur.

According to another aspect of the disclosure, a prefilled flushing syringe includes: the previously described syringe; and a predetermined volume of a fluid disposed in the barrel of the pre-filled syringe. The cap of the previously described syringe is initially in the first position, thereby preventing the fluid in the barrel from passing through the open distal end portion of the barrel to the flow channel of the connector.

According to another aspect of the disclosure, a method for expulsion of fluid from the previously described syringe includes: moving the cap of the previously described syringe from the first position to the second position, such that fluid flow from the barrel to the flow channel of the connector is permitted; attaching the connector of the cap to a vascular access device; and moving the plunger through the barrel to expel the fluid from the barrel and through the flow channel of the connector to the vascular access device.

Non-limiting illustrative examples of embodiments of the present disclosure will now be described in the following numbered clauses.

Clause 1: A syringe comprising: a barrel comprising a proximal end, a distal end comprising a distal opening, and a sidewall extending between the proximal end and the distal end; a plunger positioned in the barrel for expelling fluid from the barrel through the distal opening; and a cap connected to the distal end of the barrel comprising a connector defining a flow channel through the cap, wherein the cap is movable relative to the barrel between a first position, where fluid flow through the distal opening of the barrel to the flow channel of the connector is prevented, and a second position where fluid flow through the distal opening of the barrel to the flow channel of the connector can occur.

Clause 2: The syringe of clause 1, wherein the cap rotates relative to the barrel to move from the first position to the second position.

Clause 3: The syringe of clause 1 or clause 2, wherein the cap rotates about a longitudinal axis of the barrel to move from the first position to the second position.

Clause 4: The syringe of any of clauses 1-3, wherein the cap is configured to be manually moved from the first position to the second position.

Clause 5: The syringe of clause 4, wherein the syringe is capable of single-handed operation, both to move the cap from the first position to the second position and to expel fluid from the barrel through the flow channel of the connector by moving the plunger through the barrel.

Clause 6: The syringe of any of clauses 1-5, wherein the barrel comprises at least one of polyester, polycarbonate, polypropylene, polyethylene, polyethylene terephthalate, or acrylonitrile butadiene styrene.

Clause 7: The syringe of any of clauses 1-6, wherein the barrel further comprises an annular flange about the proximal end of the barrel for grasping the barrel and plunger to move the plunger through the barrel.

Clause 8: The syringe of any of clauses 1-7, wherein the plunger comprises a stopper positioned in the barrel and a plunger rod extending proximally from the stopper through the proximal end portion of the barrel.

Clause 9: The syringe of clause 8, wherein the stopper comprises a thermoplastic elastomer, such as isoprene.

Clause 10: The syringe of clause 8 or clause 9, wherein the stopper comprises a proximal end, a distal end, and an outer surface extending between the proximal end and the distal end that seals against an inner surface of the barrel.

Clause 11: The syringe of any of clauses 8-10, wherein the plunger rod comprises a connector portion inserted into and engaged to a cavity of the stopper for securing the stopper to the plunger rod.

Clause 12: The syringe of any of clauses 1-11, wherein the connector of the cap comprises at least one of a luer connector, a threaded connector, or a snap connector.

Clause 13: The syringe of any of clauses 1-12, wherein the connector is tapered, such that an outer diameter of a proximal end of the connector is wider than an outer diameter of a distal tip of the connector.

Clause 14: The syringe of any of clauses 1-13, further comprising a barrel seal positioned between the distal opening of the barrel and the cap, the barrel seal comprising at least one hole for fluid flow from the barrel to the flow channel of the connector.

Clause 15: The syringe of clause 14, wherein the barrel seal comprises a disk portion positioned over the distal end of the barrel and an annular wall portion extending from the disk portion positioned against an inner surface of the barrel, wherein the at least one hole extends through the disk portion.

Clause 16: The syringe of clause 15, wherein an outer surface of the annular wall portion comprise grooves configured to receive corresponding protrusions of the barrel for limiting rotation of the seal relative to the barrel.

Clause 17: The syringe of clause 16, wherein the grooves comprise axial grooves extending between a proximal end and a distal end of the annular wall of the barrel seal.

Clause 18: The syringe of any of clauses 15-17, wherein the barrel seal comprises an annular ridge extending proximally from a proximal surface of the disk portion that is received within a corresponding annular groove on the distal end of the barrel.

Clause 19: The syringe of any of clauses 15-18, wherein the barrel seal further comprises a lip extending radially inwardly from the annular wall portion of the barrel seal configured to engage the cap to retain a proximal portion of the cap within the barrel seal.

Clause 20: The syringe of any of clauses 15-19, further comprising a plug engaged to the cap, which moves as the cap moves from the first position to the second position, wherein, when the cap is in the first position, the plug is over the at least one hole of the barrel seal preventing fluid flow through the at least one hole of the barrel seal to the flow channel of the connector, and when the cap is in the second position, the plug is spaced apart from the at least one hole of the barrel seal to allow fluid flow from the barrel to the flow channel of the connector through the at least one hole of the barrel seal.

Clause 21: The syringe of clause 20, wherein the plug comprises a sealing portion that moves over the at least one hole of the barrel seal and at least one arm extending from the scaling portion, which is engaged to a portion of the cap.

Clause 22: The syringe of clause 20 or clause 21, wherein an end of the at least one arm of the plug is pivotally connected to an inner surface of the cap so that the plug can move to uncover the at least one hole of the barrel seal as the cap moves from the first position to the second position.

Clause 23: The syringe of any of clauses 20-22, wherein the cap comprise at least one pair of protrusions extending radially inwardly from an inner surface of the cap, and wherein an end of the at least one arm of the plug is positioned between the pair of protrusions, such that the protrusions move the plug as the cap rotates from the first position to the second position.

Clause 24: The syringe of any of clauses 20-23, wherein the plug further comprises a post inserted into a hole of the disk portion of the seal which forms a rotation point as the cap moves from the first position to the second position.

Clause 25: The syringe of any of clauses 20-24, wherein the plug comprises an elastomer seal configured to be positioned over the at least one hole of the barrel seal for sealing the at least one hole of the barrel seal when the cap is in the first position.

Clause 26: The syringe of clause 25, wherein the plug further comprises a body comprising a rigid thermoplastic material, and wherein the elastomer seal is mounted or adhered to the body of the plug.

Clause 27: The syringe of any of clauses 14-26, wherein the at least one hole of the barrel seal is axially aligned with the distal opening of the barrel, and wherein a diameter of the at least one hole of the barrel seal is smaller than a diameter of the distal opening of the barrel.

Clause 28: The syringe of any of clauses 14-27, wherein the barrel seal comprises a thermoplastic elastomer material.

Clause 29: The syringe of any of clauses 14-28, wherein the barrel seal remains stationary as the cap moves from the first position to the second position.

Clause 30: The system of any of clauses 1-29, wherein the cap further comprises a shield extending about the connector, the shield comprising an inner surface comprising threads configured to engage threads of a female connector for securing the syringe and the cap to the female connector.

Clause 31: The syringe of clause 30, wherein a distal end of the connector protrudes beyond a distal end of the shield.

Clause 32: The syringe of clause 30 or clause 31, wherein a direction of rotation for connecting the syringe to the female connector is opposite a direction of rotation to move the cap from the first position to the second position.

Clause 33: The syringe of any of clauses 30-32, wherein the barrel further comprises an extension portion extending distally from the distal end of the barrel, and wherein the cap is connected to and at least partially enclosed by the extension portion.

Clause 34: The syringe of clause 33, wherein the extension portion comprises a proximal end extending from the distal end of the barrel, an open distal end, and an annular sidewall extending between the proximal end and the distal end.

Clause 35: The syringe of clause 33 or clause 34, wherein the sidewall of the barrel comprises a tapered portion between the distal end of the barrel and other portions of the barrel for guiding fluid in the barrel towards the distal opening of the barrel.

Clause 36: The syringe of any of clauses 33-35, wherein the cap comprises at least one tab extending radially outward from the shield for rotating the cap relative to the barrel.

Clause 37: The syringe of any of clauses 33-36, wherein the cap further comprises a rotation ring comprising the at least one tab, wherein the rotation ring extends about the shield of the cap and the extension portion of the barrel, and wherein the rotation ring is configured to be rotated by a user to move the cap between the first position and the second position.

Clause 38: The syringe of clause 37, wherein the cap comprises multiple tabs extending radially outwardly from the shield, and wherein the multiple tabs are connected to the rotation ring at different positions about a circumference of the ring to support the rotation ring.

Clause 39: The syringe of any of clauses 36-38, wherein the extension portion comprises at least one slot, and wherein the at least one tab of the cap extends through the slot so that the at least one tab can be grasped by the user to rotate the cap relative to the barrel.

Clause 40: The syringe of clause 39, wherein the at least one slot extending from a distal end of the extension portion comprises an axial portion, for inserting the cap into the extension portion, and a circumferential portion, for rotating the cap relative to the barrel between the first position and the second position.

Clause 41: The syringe of clause 39 or clause 40, wherein the at least one slot comprises at least one bump protruding from a surface of the slot, which restricts the at least one tab from sliding through the slot for locking the cap in the extension portion.

Clause 42: The syringe of any of clauses 33-41, wherein the extension portion further comprises protrusions on an inner surface of the extension portion for restricting rotation of a barrel seal positioned over the distal opening of the barrel.

Clause 43: The syringe of clause 42, wherein the protrusions comprise axially extending ridges, ribs, or columns on the inner surface of the extension portion.

Clause 44: A prefilled flushing syringe, comprising: the syringe of any of clauses 1-43; and a predetermined volume of a fluid disposed in the barrel of the pre-filled syringe, wherein the cap is initially in the first position, thereby preventing the fluid in the barrel from passing through the open distal end portion of the barrel to the flow channel of the connector.

Clause 45: The prefilled syringe of clause 44, wherein the fluid comprises a saline flush solution.

Clause 46: The prefilled syringe of clause 44 or clause 45, wherein the fluid comprises a therapeutic agent.

Clause 47: The prefilled syringe of any of clauses 44-46, wherein the barrel contains from about 1 mL to about 50 mL of the fluid.

Clause 48: A method for expulsion of fluid from the syringe of any of clauses 1-43, the method comprising: moving the cap from the first position to the second position, such that fluid flow from the barrel to the flow channel of the connector is permitted; attaching the connector of the cap to a vascular access device; and moving the plunger through the barrel to expel the fluid from the barrel and through the flow channel of the connector to the vascular access device.

Clause 49: The method of clause 48, wherein the fluid contained in the barrel of the syringe comprises a saline flush solution.

Clause 50: The method of clause 48 or clause 49, wherein the fluid contained in the barrel of the syringe comprises a therapeutic agent.

Clause 51: The method of any of clauses 48-50, wherein the barrel contains from about 1 mL to about 50 mL of the fluid.

Clause 52: The method of any of clauses 48-51, wherein the cap is manually moved from the first position to the second position by grasping a portion of the cap and rotating the cap relative to a longitudinal axis of the syringe.

Clause 53: The method of clause 52, wherein the cap is manually moved from the first position to the second position by grasping a portion of the cap with one hand.

Clause 54: The method of any of clauses 48-53, further comprising advancing the plunger through the barrel by a small amount prior to attaching the syringe to the VAD to remove air from an interior of the barrel.

Clause 55: The method of any of clauses 48-54, wherein attaching the cap to the VAD comprises inserting a distal tip of the connector through a septum of a fluid port of the VAD and twisting the syringe, such that threads of the syringe engage corresponding threads of a female connector of the VAD.

Clause 56: The method of clause 55, wherein the cap is moved from the first position to the second position by rotating the cap relative to the barrel in a first direction, and the cap is attached to the VAD by rotating the syringe in a second direction opposite from the first direction.

Clause 57: The method of any of clauses 48-56, wherein the connector comprises a male luer connector and the cap is attached to a female luer connector of the VAD by inserting the male luer connector into the female luer connector.

### BRIEF DESCRIPTION OF THE DRAWINGS

FIG. 1A is a perspective view of a syringe comprising an integrated cap, according to an aspect of the present disclosure.

FIG. 1B is an exploded view of the syringe and cap of FIG. 1A.

FIG. 2A is a perspective view of a barrel of the syringe of FIG. 1A, according to an aspect of the present disclosure.

FIG. 2B is a front view of a distal end portion of the barrel of FIG. 2A.

FIG. 2C is a perspective view of a cross-section of the distal end portion of the barrel of FIG. 2A.

FIG. 3A is a perspective view of a distal surface of a seal of the syringe of FIG. 1A, according to an aspect of the disclosure.

FIG. 3B is a perspective view of a proximal surface of the seal of FIG. 3A.

FIG. 3C is a perspective view of a cross-section of the distal end portion of the syringe barrel showing the seal seated proximate to the distal end of the barrel, according to an aspect of the disclosure.

FIG. 4A is a perspective view of a distal portion of the cap of FIG. 1A, according to an aspect of the present disclosure.

FIG. 4B is a perspective view of a proximal portion of the cap of FIG. 4A.

FIG. 4C is a perspective view of a cross-section of the cap of FIG. 4A.

FIG. 4D is a perspective view of a cross-section of the distal end portion of the syringe barrel showing the seal seated proximate to the distal end of the barrel and the cap seated against the seal, according to an aspect of the disclosure.

FIG. 5A is a perspective view of a plug for preventing fluid flow from the barrel to the cap of the syringe of FIG. 1A, according to an aspect of the present disclosure.

FIG. 5B is a bottom view of the plug of FIG. 5A.

FIG. 6A is a perspective view of a cross-section of the syringe of FIG. 1A showing the seal, plug, and cap connected to the distal end of the barrel, with the plug in a closed position, according to an aspect of the present disclosure.

FIG. 6B is a cross-sectional view of the syringe of FIG. 1A showing the seal, plug, and cap connected to the distal end of the barrel, with the plug in the closed position, according to an aspect of the present disclosure.

FIG. 6C is a perspective view of a cross-section of the distal end of the syringe of FIG. 1A showing the plug in the closed position engaged by a portion of the cap, according to an aspect of the present disclosure.

FIG. 7A is a perspective view of a cross-section of the syringe of FIG. 1A showing the seal, plug, and cap connected to the distal end of the barrel, with the plug in an open position, according to an aspect of the present disclosure.

FIG. 7B is another cross-sectional view of the syringe of FIG. 1A showing the seal, plug, and cap connected to the distal end of the barrel, with the plug in the open position, according to an aspect of the present disclosure.

FIG. 7C is a cross-sectional view of the distal end of the syringe of FIG. 1A showing the plug in the open position engaged by a portion of the cap, according to an aspect of the present disclosure.

FIG. 8 is a flow chart showing steps for using a syringe comprising an integrated cap for delivering a medical fluid to a patient through a vascular access device, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped. manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to syringes 10, such as prefilled syringes, configured to be used by a practitioner for delivering a medical fluid, to a patient through, for example, a vascular access device (VAD). The practitioner can be, for example, a medical technician, nurse, physician assistant, physician, or other trained or untrained clinicians or medical caregivers. The medical fluid can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringe 10 of the present disclosure. The medical fluid can also be a medication, a total parenteral nutrient (TPN) liquid, or another therapeutic agent used for treatment of chronic or acute conditions, as are known in the art. Exemplary therapeutic agents can include, for example, drugs, chemicals, biological or biochemical substances that, when delivered in a therapeutically effective amount to the patient, achieve a desired therapeutic effect.

As previously described, the syringes 10 of the present disclosure are intended to reduce contamination risks and to ensure that the distal tip of the syringe barrel remains sterile and free from contamination prior to and while it is connected to the VAD. Further, the syringes 10 of the present disclosure are designed for easy operation and, in particular, to avoid difficulties with removing tip caps of conventional prefilled syringes. In some particularly advantageous examples, the syringes 10 of the present disclosure can be operated by a practitioner with one hand. Specifically, the practitioner is able to open and close the syringe cap with one hand by, for example, pressing, sliding, pulling or otherwise manipulating a tab of a syringe cap to open and close the cap, as described in further detail herein. By contrast, as previously described, for conventional prefilled syringes, a practitioner needs to grasp the syringe barrel with one hand and grasp the tip cap with the other hand in order to pull the tightly fitted tip cap away from the distal tip of the syringe barrel. Once the syringe cap is opened, the practitioner is able to expel the medical fluid from the syringe barrel of the syringe 10 with one hand by, for example, pushing a plunger or stopper through the syringe barrel.

In some examples, the syringes 10 of the present disclosure are provided as a prefilled syringe containing the flush solution or other medical fluid. The prefilled syringe 10 can also include caps, clips, retainers, and/or other packaging to hold the plunger rod in place and to ensure that the flush solution or another medical fluid does not leak from the prefilled syringe 10 at unexpected times, such as during transport.

### Syringe With Integrated Cap

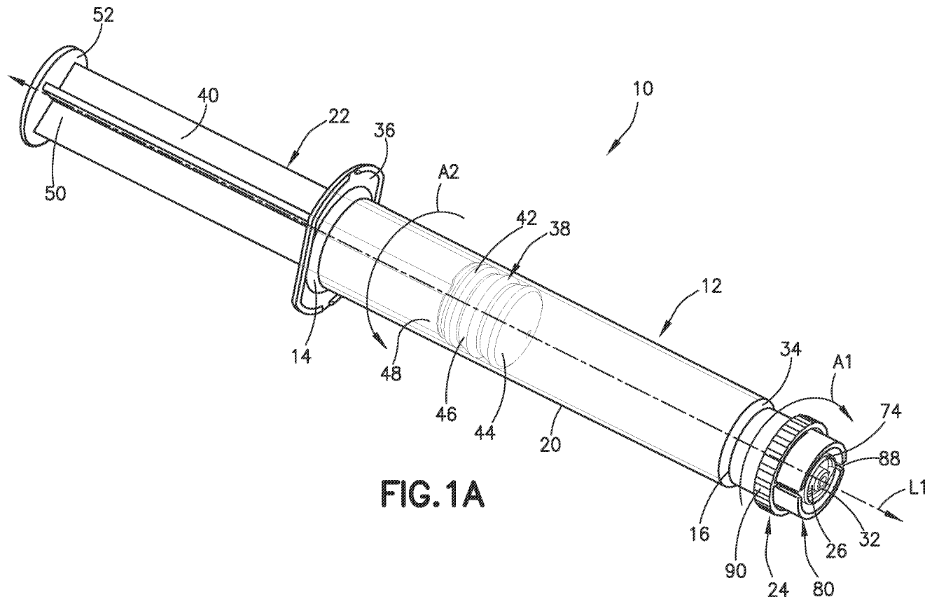

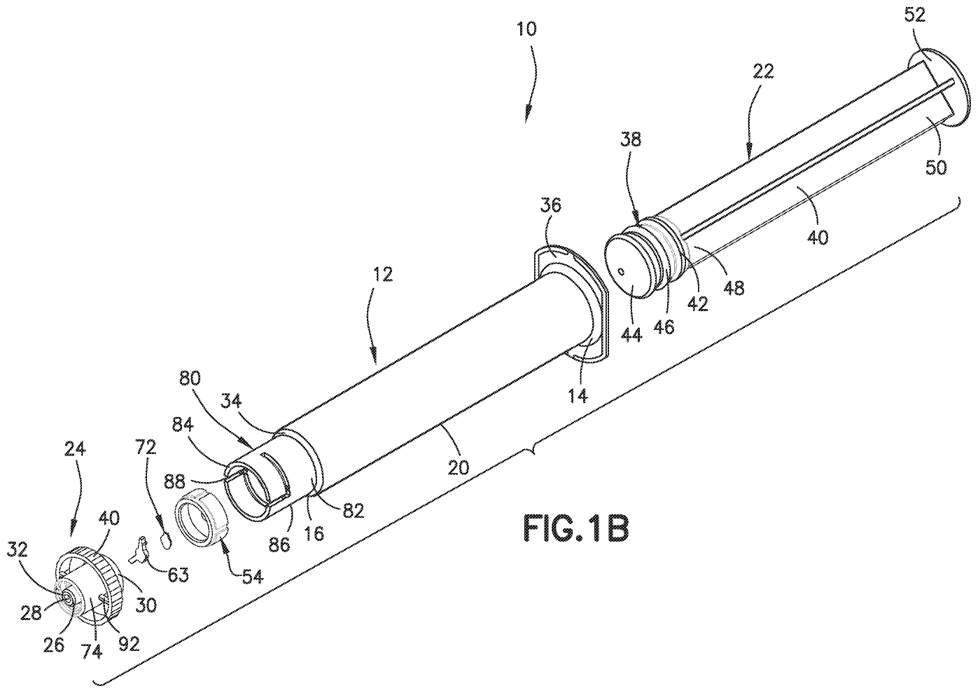

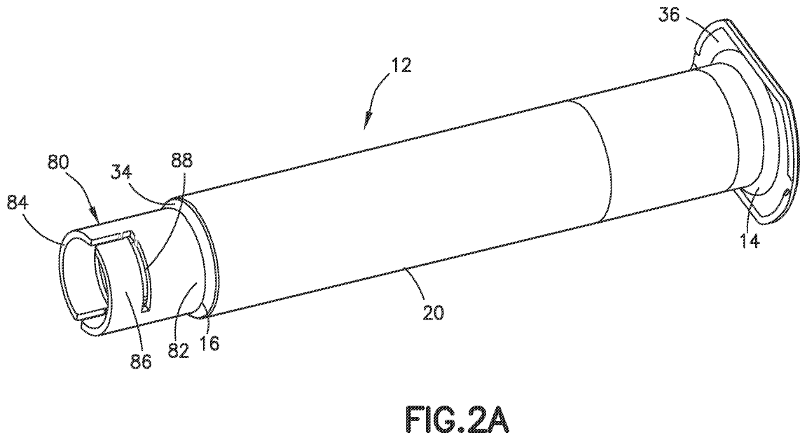

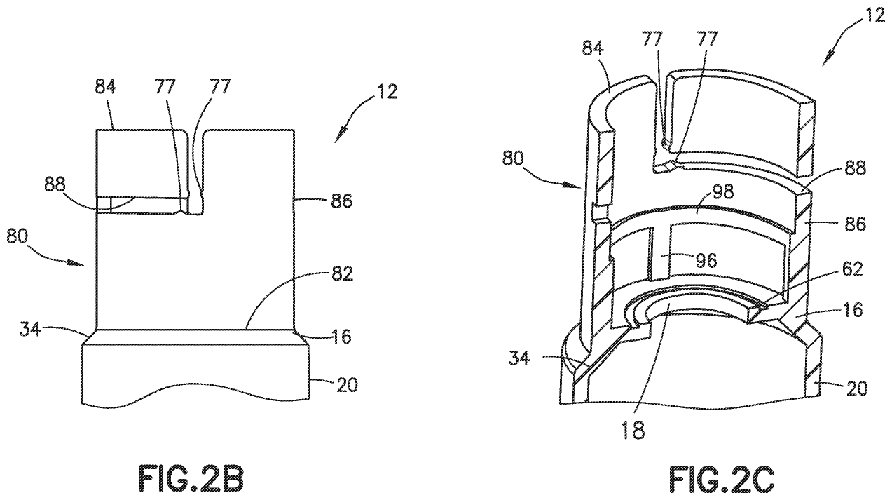

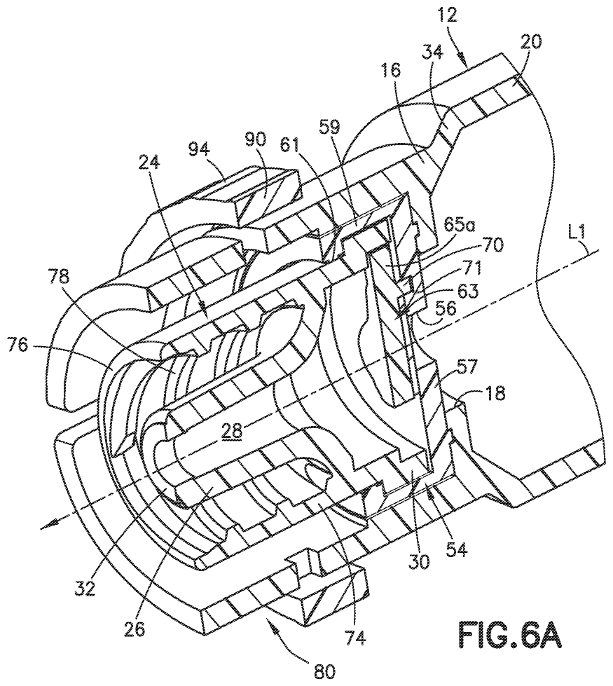

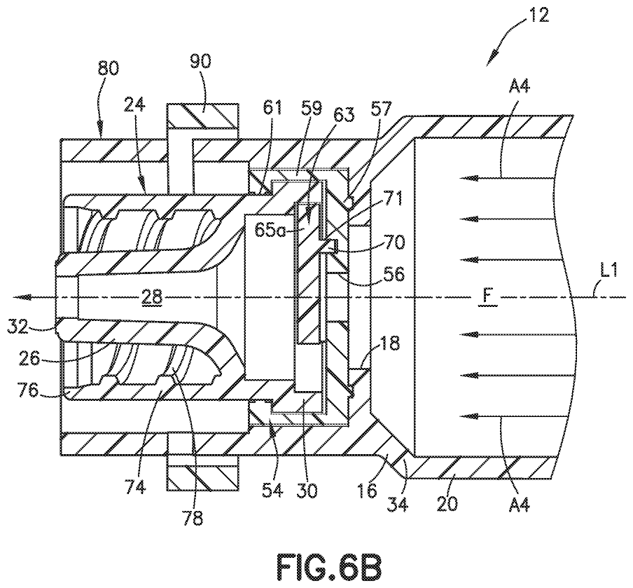

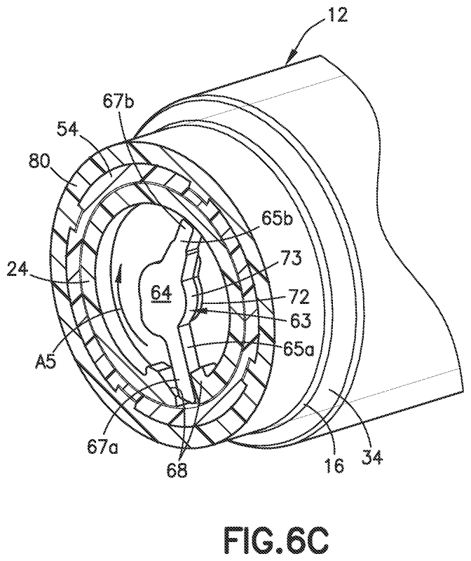

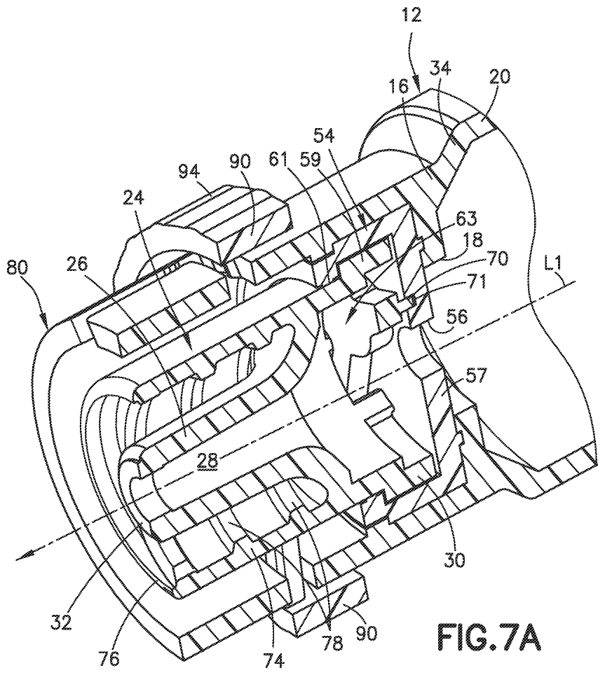

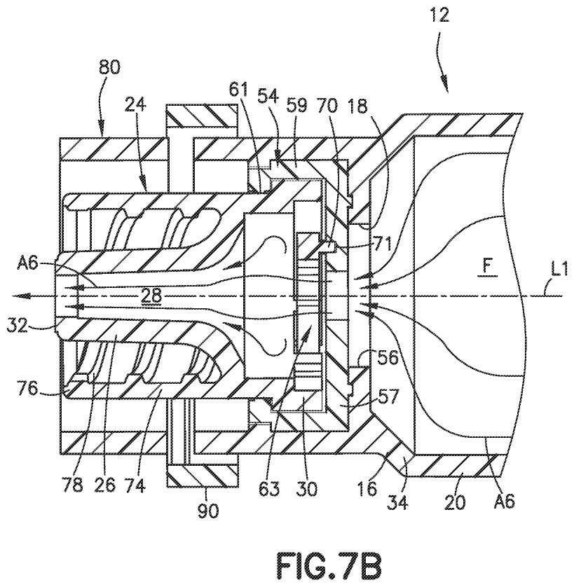

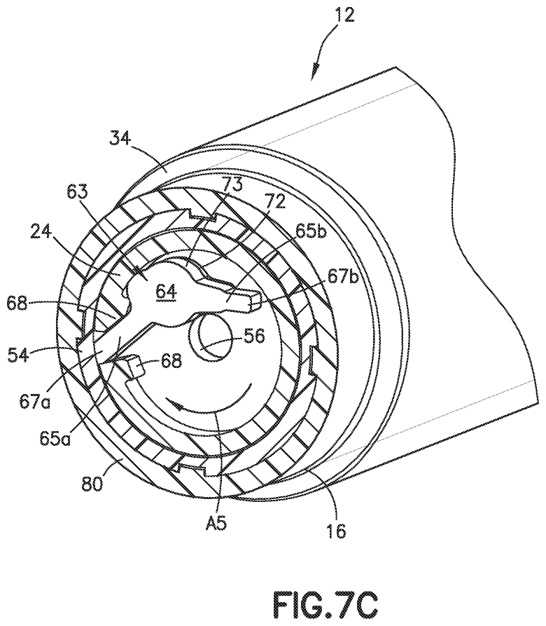

With reference to FIGS. 1A and 1B, the syringe 10 of the present disclosure comprises a barrel 12, which can comprise a proximal end 14, a distal end 16 comprising a distal opening 18 (shown most clearly in FIG. 2C), and a sidewall 20 extending between the proximal end 14 and the distal end 16. The syringe 10 further comprises a plunger 22 positioned in the barrel 12 for expelling the medical fluid F (shown in FIGS. 6A, 6B, 7A, and 7B) from the barrel 12 through the distal opening 18. The syringe 10 further comprises a cap 24, valve, cover, sealing arrangement, or similar structure connected to the distal end 16 of the barrel 12 for sealing the medical fluid F within the barrel 12 and for guiding fluid flow from an interior of the barrel 12 to expel the medical fluid F from the barrel 12. In some examples, the cap 24 comprises a connector 26 or stem extending distally from the distal end 16 of the barrel 12. The connector 26 can be an elongated and/or tubular member defining a flow passage or flow channel 28 extending from a proximal end 30 of the cap 24 to a distal end or distal tip 32 of the connector 26. As described in further detail herein, the connector 26 can be a male needleless connector 26 that is configured to be inserted into a female needless connector, such as a female connector that forms a fluid port or opening of a VAD. For example, the cap 24 can be configured to be used with female needleless connectors, such as female luer connectors, for a range of dimensions permitted by various design protocols, such as ISO 80369-7:2016 or ISO 80369-7:2021 (Female Luer Lock Connector dimensions). In some particular examples, the cap 24 can be sized to engage female needleless connectors having threads with a width at a crest of each thread of from about 0.3 mm to about 1.0 mm and a width at a root of the thread from about 0.5 mm to about 1.2 mm.

When the connector 26 is inserted into the female connector, fluid communication is established between the VAD and the interior of the barrel 12 through the cap 24. Further, the cap 24 is movable relative to the barrel 12 between a closed or first position (shown in FIGS. 6A-6C), where fluid flow through the distal opening 18 of the barrel 12 to the flow channel 28 of the connector 26 is prevented, and an open or second position (shown in FIGS. 7A-7C), where fluid flow through the distal opening 18 of the barrel 12 and the flow channel 28 of the connector 26 to the VAD can occur. For example, the cap 24 can be configured to twist or rotate about a longitudinal axis L1 (shown in FIGS. 1A, 6A, 6B, 7A, and 7B) of the barrel 12 and syringe 10, which moves the cap 24 from the closed position (shown in FIGS. 6A-6C) to the open position (shown in FIGS. 7A-7C).

In some examples, the cap 24 is configured to be manually movable, meaning that the practitioner can grasp a portion of the cap 24 and rotate, twist, slide, or otherwise move the cap 24 from the closed position to the open position. Further, as previously described, the cap 24 can be configured for single-handed operation. In particular, the practitioner desirably is able both to move the cap 24 from the closed position to the open position and to expel the medical fluid F from the barrel 12 through the flow channel 28 of the connector 26 by moving the plunger 22 through the barrel 12 using one hand.

With continued reference to FIGS. 1A and 1B, as well as FIGS. 2A-2C, the barrel 12 can be a conventional fluid-delivery syringe barrel used for medical procedures made by, for example, an injection molding process. For example, the barrel 12 can be substantially similar in shape, size, and configuration to barrels of syringes used for administering a flush solution to a VAD, as are known in the art. In some examples, the barrel 12 can be a cylindrical or elliptical prism structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels 12 for flush syringes similar to the barrel 12 of the present disclosure are described, for example, in U.S. Patent Appl. Pub. No. 2020/0061297, entitled “Flush Syringe Assembly with Controlled Pulsatile Flushing.” which is incorporated herein by reference in its entirety. Dimensions of the syringe barrel 12 can be determined based on the type of fluid injection being performed. In particular, the barrel 12 can be configured to contain a variety of fluid volumes depending upon the type and amount of fluid being delivered from the barrel 12 to the patient. For example, the barrel 12 can define an interior that contains about 1 mL to about 50 ml of the medical fluid or, preferably, from about 5 mL to 20 mL of the medical fluid.

In some examples, the sidewall 20 of the barrel 12 comprises a tapered portion 34 (shown in FIGS. 2A-2C) proximate to the distal end 16 of the barrel 12. For example, the tapered portion 34 can be positioned between the distal end 16 of the barrel 12 and other portions of the sidewall 20 of the barrel 12. The tapered portion 34 can be configured to guide fluid in the barrel 12 towards the distal opening 18 of the barrel 12 for expelling the medical fluid in the barrel 12 through the distal opening 18 towards the flow passage or flow channel 28 of the connector 26. The tapered portion 34 can be shaped to reduce dead space having a sloped or angled surface corresponding with a distal surface of a stopper or plunger 22, which moves through the interior of the barrel 12, to ensure that the fluid moves through the barrel 12 to the distal opening 18. Reducing or eliminating dead space between the stopper or plunger 22 and the distal end 16 of the barrel 12 helps to ensure that the appropriate or expected amount of fluid is expelled from the barrel 12 and delivered to the patient. In some examples, the barrel 12 further comprises a finger flange 36 or grip. For example, as shown in FIGS. 1A and 1B, the finger flange 36 can extend about the proximal end 14 of the barrel 12 for grasping the barrel 12 and plunger 22 to move the plunger 22 through the barrel 12.

With continued reference to FIGS. 1A and 1B, the syringe 10 further comprises the plunger 22 configured to move through an interior of the syringe barrel 12 for aspirating a medical fluid into the interior of the syringe barrel 12 (if the syringe 10 is not a prefilled syringe 10) and/or for expelling the fluid F from the barrel 12 through the flow passage or flow channel 28 of the connector 26. The plunger 22 can comprise a stopper 38 and a plunger rod 40 connected to and extending from the stopper 38 and through the proximal end 14 of the barrel 12. The stopper 38 can have many features of conventional syringe stoppers or plungers, as are known in the art. For example, the stopper 38 can comprise a thermoplastic elastomer material, such as polypropylene or polyethylene, as well as from synthetic or natural rubber (e.g., isoprene). Further, the stopper 38 can comprise a proximal surface 42 or proximal end. a distal surface 44 or end, and an outer peripheral surface 46 extending between the proximal surface 42 and the distal surface 44. The outer peripheral surface 46 can be configured to seal against an inner surface of the sidewall 20 of the barrel 12 for moving the medical fluid F through the barrel 12. In some examples, the distal end or distal surface 44 of the stopper 38 can be tapered having a slope or angle that matches the tapered portion 34 of the sidewall 20 of the barrel 12, as previously described.

The plunger 22 also includes the plunger rod 40 connected to the stopper 38. The plunger rod 40 can be, for example, an injection molded part formed from a rigid thermoplastic material, such as polyester, polycarbonate, polypropylene, polyethylene, polyethylene terephthalate, or another thermoplastic material, as are known in the art. The plunger rod 40 can include a distal end 48 engaged to the stopper 38. For example, the distal end 48 of the plunger rod 40 can include a connector (not shown) that is inserted into a corresponding cavity or slot on the proximal surface 42 of the stopper 38. The plunger rod 40 can also include a proximal end 50 protruding proximally through the proximal end 14 of the barrel 12 and a body extending between the proximal end 50 and the distal end 48 of the plunger rod 40. The proximal end 50 of the plunger rod 40 can include a thumb press plate 52 for manipulating the plunger rod 40 to move the plunger rod 40 and stopper 38 through the syringe barrel 12. The body of the plunger rod 40 can have a variety of cross-sectional shapes and configurations within the scope of the present disclosure. For example, the body can have a generally cross shaped cross-section. In other examples, the cross-section of the plunger rod 40 can be an I-beam shape, hollow circle, hollow square, hollow rectangle, or L-shaped.

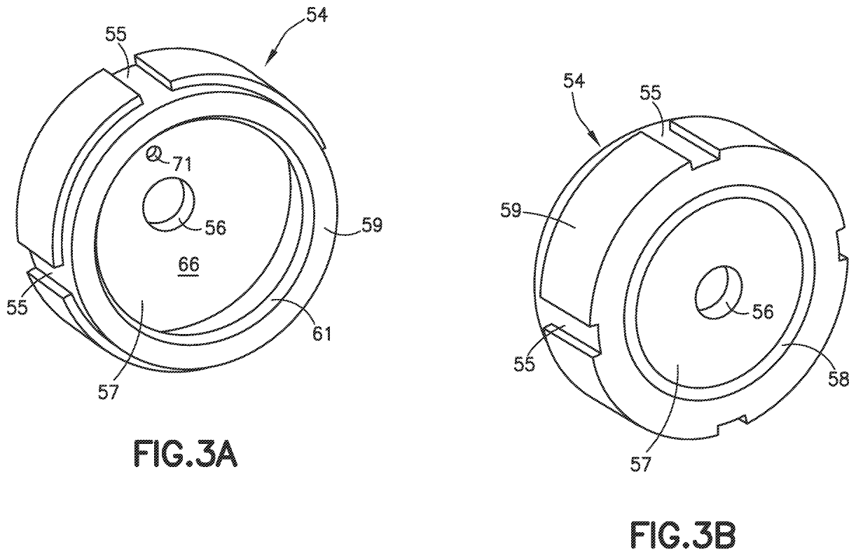

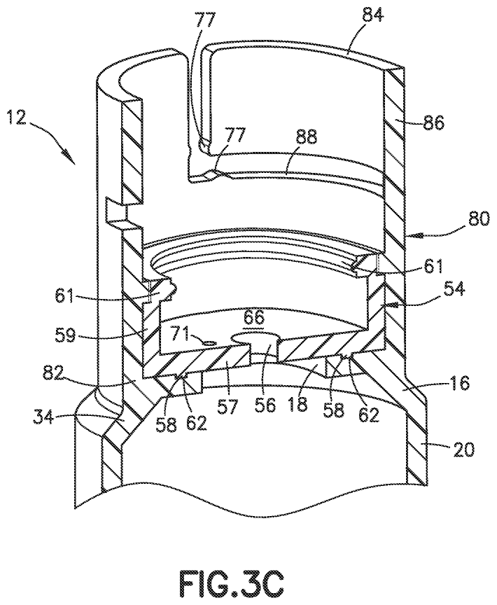

With continued reference to FIGS. 1A and 1B, as well as FIGS. 3A-3C, the syringe 10 further comprises a barrel seal 54 or gasket for sealing the distal opening 18 of the barrel 12 to ensure that fluid passing through the distal opening 18 of the barrel 12 passes to the flow passage or flow channel 28 of the connector 26. As shown in FIG. 3C, the barrel seal 54 can be positioned proximate to the distal opening 18 of the barrel 12 between the distal end 16 of the barrel 12 and the cap 24. The barrel seal 54 can be formed from an elastomeric material, such as silicone, rubber, isoprene, or a similar flexible elastomer, that seals the distal end 16 and distal opening 18 of the barrel 12.

In some examples, the barrel seal 54 comprises substantially flat sheet or disk portion 57 positioned over the distal end 16 of the barrel 12 and an annular wall portion 59 extending distally from the disk portion 57 and positioned against an annular inner surface of a portion of the barrel 12. The barrel seal 54 also includes an opening, perforations, or fluid port, such as a through-hole 56, extending through the disk portion 57 that allows fluids to flow from the barrel 12 to the flow channel 28 of the connector 26.

As shown in FIG. 3C, the hole 56 of the barrel seal 54 can be axially aligned with the distal opening 18 of the barrel 12 along the longitudinal axis L1 (shown in FIGS. 1A, 6A, 6B, 7A, and 7B) of the syringe 10, meaning that the axis L1 passes through a center of the hole 56 and a center of the distal opening 18. As shown, for example, in FIG. 3C, the hole 56 of the barrel seal 54 can be smaller (e.g., can have a smaller diameter and/or area) than the distal opening 18 of the barrel 12. In some examples, the barrel seal 54 also includes structures for maintaining position of the barrel seal 54 over the distal opening 18 of the barrel 12. For example, the barrel seal 54 can include a proximal ridge 58 or O-ring extending from a proximal surface of the barrel seal 54, which is positioned in a corresponding groove 62 of the distal end 16 of the barrel 12. The engagement between the ridge 58 or O-ring and the groove 62 can ensure that the barrel seal 54 remains in place and does not slide relative to distal end 16 or distal opening 18 of the barrel 12. The ridge 58 can also seal the distal opening 18 preventing fluid from collecting in a space between the distal end 16 of the barrel 12 and the barrel seal 54.

With reference to FIGS. 3A-3C, the barrel seal 54 can also include a lip 61 or inwardly directed ridge extending radially inwardly from the annular wall portion 59 of the barrel seal 54. The lip 61 can be positioned to engage the cap 24 to retain a proximal portion of the cap 24 within the barrel seal 54. Accordingly, the engagement between the lip 61 and the cap 24 ensures that the cap 24 remains seated in the barrel seal 54, limiting distal movement of the cap 24 away from the barrel seal 54 as the cap 24 rotates between the closed position and the open position. It is noted, however, that, in some examples, the cap 24 may lift away from the barrel seal 54 slightly as the cap 24 moves from the closed position to the open position creating a gap between the disk portion 57 of the barrel seal 54 and the cap 24. However, the width of the gap between the barrel seal 54 and the cap 24 is limited by the engagement between the cap 54 and the lip 61.

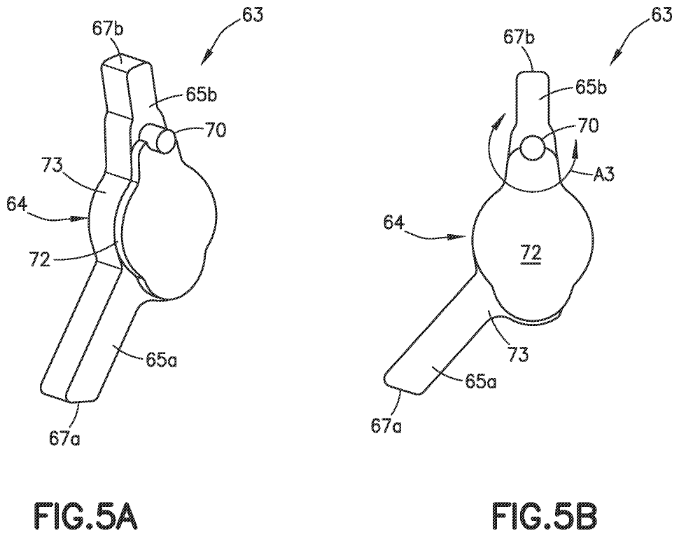

With reference to FIGS. 1A and 1B, as well as FIGS. 5A and 5B, the syringe 10 further comprises a plug 63 or valve engaged to the cap 24. The plug 63 is configured to move away from the hole 56 of the barrel seal 54 as the cap 24 moves from the closed position to the open position. In particular, when the cap 24 is in the closed position (shown in FIGS. 6A-6C), the plug 63 is over the hole 56 of the barrel seal 54 preventing fluid flow through the hole 56 to the flow passage or flow channel 28 of the connector 26. When the cap 24 is in the open position, the plug 63 is at least partially spaced apart from the hole 56 of the barrel seal 54 to allow fluid flow from the barrel 12 to the flow passage or flow channel 28 of the connector 26 through the hole 56.

In some examples, the plug 63 comprises a sealing portion 64 sized to move over and cover the hole 56 for preventing fluid flow through the hole 56. The plug 63 also includes one or more elongated members or arms 65a. 65b extending from the scaling portion 64, which can be configured to engage or be connected to a portion of the cap 24 to rotate the plug 63, as shown by arrow A5 (in FIGS. 6C and 7C). For example, as shown in FIGS. 5A and 5B, the plug 63 includes a first arm 65a extending from the sealing portion 64 in a first direction and a second arm 65b extending from the sealing portion 64 in a second direction. The arms 65a, 65b of the plug 63 can include free ends 67a, 67b, which can contact and/or can be pivotally connected to an inner surface of the cap 24. Due to the engagement between one or both of the free ends 67a, 67b and the cap 24, movement of the cap 24 causes the plug 63 to move to uncover the hole 56 of the barrel seal 54.

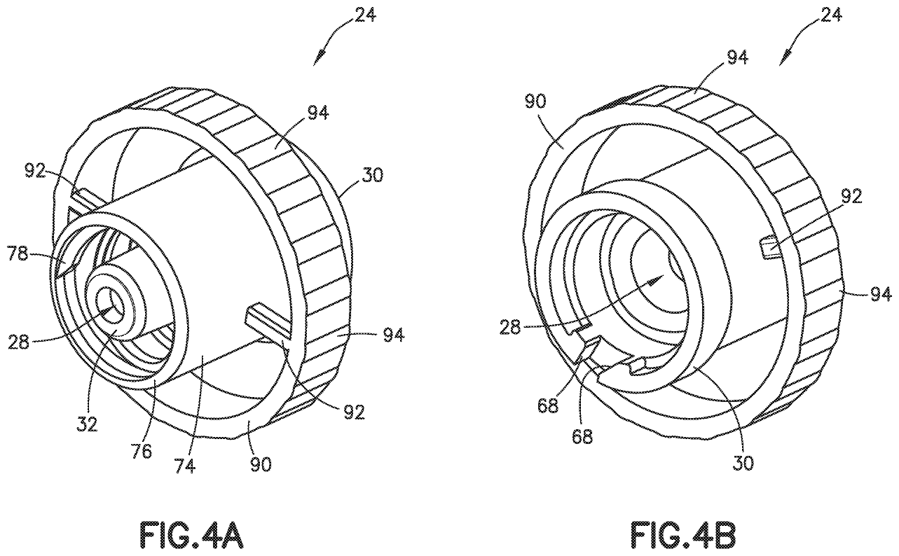

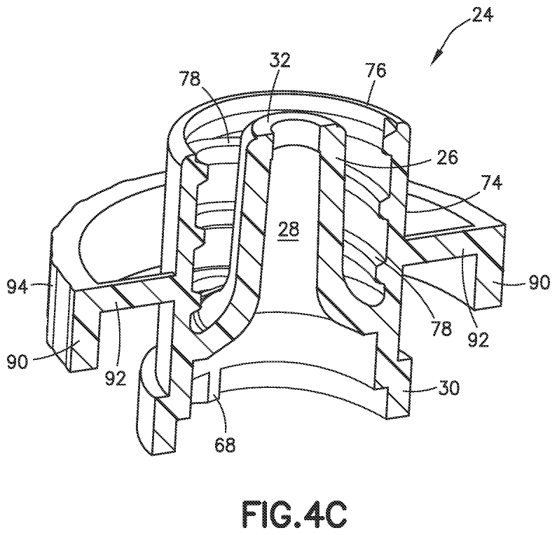

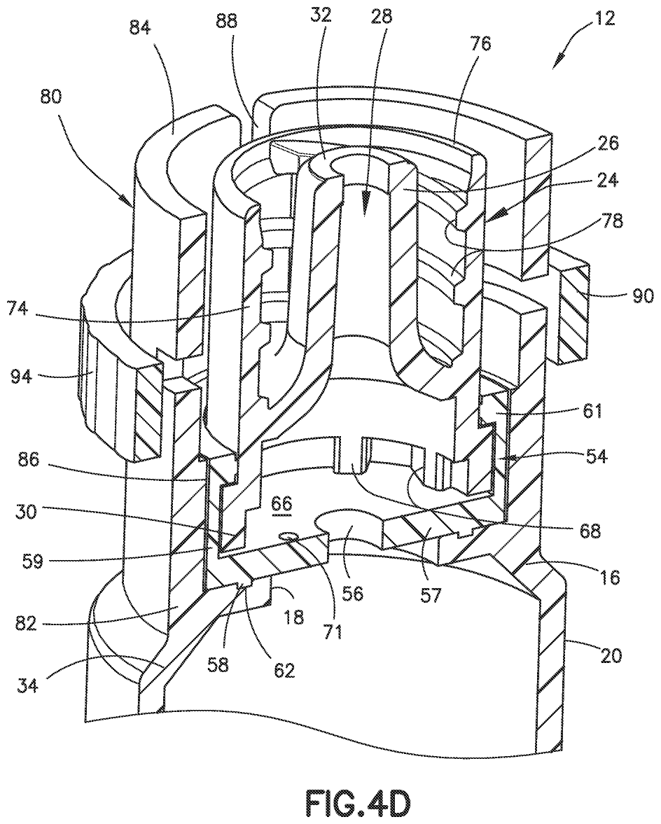

In some examples, the cap 24 can comprise structures, such as protrusions 68, ribs, slots, notches, gaps, or other structures, for contacting and transferring movement of the cap 24 to the plug 63. For example, as shown in FIGS. 4B, 6C, and 7C, the cap 24 includes a pair of protrusions 68. The free end 67a of the first arm 65a is positioned between the protrusions 68, so that rotation of the cap 24 is transferred to the plug 63, which causes the plug 63 to move away from the hole 56 as the cap 24 moves from the closed position to the open position. The plug 63 can also include a post 70 or pivot point, which is received within a hole 71 on the distal surface 66 of the barrel seal 54. The plug 63 is configured to rotate about the post 70 (as shown by arrow A3 in FIG. 5B) or pivot point, which moves the sealing portion 64 of the plug 63 towards or away from the hole 56.

With continued reference to FIGS. 5A and 5B, the plug 63 can be formed from separate parts that are adhered together to form the plug 63. For example, the plug 63 can comprise an elastomer seal 72 on the sealing portion 64 of the plug 63, which is configured to be positioned over the hole 56 of the barrel seal 54 for sealing the hole 56 of the barrel seal 54 when the cap 24 is in the closed position. The elastomer seal 72 can be formed from an elastomeric material, such as silicone, polypropylene, polyethylene, isoprene, or natural rubber. The plug 63 further comprises a body 73, which can comprise a more rigid material, such as a rigid thermoplastic material, for supporting the elastomer seal 72. In particular, the body 73 desirably is sufficiently rigid to rotate and move towards or away from the hole 56 of the barrel seal 54 as the cap 24 moves or rotates.

With reference to FIGS. 1A and 1B, as well as FIGS. 4A and 4B, the syringe 10 further comprises the cap 24 connected to the distal end 16 of the barrel 12 and received within the barrel seal 54. For example, as described in further detail herein, the barrel 12 can include locking structures, such as slots, grooves, detects, protrusions, ridges, or similar structures, for providing a mechanical engagement between the cap 24 and the barrel 12, thereby securing the cap 24 to the barrel 12 proximate to the distal opening 18 of the barrel 12. The cap 24 comprises the stem or connector 26 defining the flow passage or flow channel 28, which permits fluid flow from the barrel 12 through the connector 26. As previously described, in some examples, the connector 26 of the cap 24 comprises a male luer connector. As used herein, a “luer connector” refers to a connector that includes a tapered portion (i.e., a luer taper) for creating a friction engagement between the connector 26 and a tapered cavity, such as a cavity of a female luer connector, configured to receive and engage the tapered connector 26. For example, the connector 26 can be a tapered elongated structure where an outer diameter of a proximal end of the connector 26 is wider than an outer diameter of the distal tip 32 of the connector 26. The connector 26 can be configured to be connected directly or indirectly to a fluid port, valve, or another terminal access portion of a VAD comprising a female luer connector. For example, a common type of fluid port of a VAD is a pierceable septum or pre-slit septum made of rubber or another elastomeric material, which permits insertion of a blunt elongated member or frusto-conically shaped tip, such as the distal tip 32 of the connector 26, in order to infuse fluids or to withdraw fluids from a catheter of the VAD. The connector 26 of the present disclosure can be sized to be inserted through the septum of the VAD, which establishes fluid communication between the barrel 12 and the VAD through the connector 26.

In some examples, the cap 24 further comprises a shield 74 extending about the connector 26. The shield 74 can be an annular or tubular structure having a proximal end connected to a proximal end of the connector 56 and an open distal end 76 that encloses or surrounds the connector 26. The shield 74 comprises an inner surface with threads 78 configured to engage threads of a female connector for securing the syringe 10 and cap 24 to the female connector. As previously described, the threads 78 of the shield 74 can be configured to mate to a female connector with different thread arrangements, such as a female connector with threads with a width at a crest of each thread of from about 0.3 mm to about 1.0 mm and a width at a root of the thread from about 0.5 mm to about 1.2 mm.

The distal tip 32 or end of the connector 26 can extend distally beyond the open distal end 76 of the shield 74, such that the distal end or tip 32 of the connector 26 can be inserted through a cover or septum over an opening or fluid port of the female connector. In some examples, the cap 24 can be configured to rotate in a first direction (shown by arrow A1 in FIG. 1A) in order to move the cap 24 from the closed position to the open position. The threads 78 on the inner surface of the shield 74 can be arranged such that the cap 24 and syringe 10 are rotated in an opposite or second direction (shown by arrow A2 in FIG. 1A) in order to secure the cap 24 and the syringe 10 to the female connector for expelling fluid from the syringe 10 to the VAD.

With continued reference to FIGS. 1A and 1B, as well as FIGS. 2A-2C, the barrel 12 can further comprise structures for securing the cap 24 over the distal opening 18 of the barrel 12 so that fluid flows through the distal opening 18 to the flow passage or flow channel 28 of the connector 26. For example, the barrel 12 can comprise an extension portion 80 extending distally from the distal end 16 of the barrel 12. As shown in FIGS. 1A and 1B, the cap 24 is connected to and at least partially enclosed by the extension portion 80. The extension portion 80 can be an annular or tubular structure comprising a proximal end 82 extending from the distal end 16 of the barrel 12, an open distal end 84, and an annular sidewall 86 extending between the proximal end 84 and the distal end 86. In some examples, the cap 24 can be inserted or press fit into the extension portion 80 through the open distal end 84 of the extension portion 80. The extension portion 80 can be integrally formed with other portions of the barrel 12 or can be a separate part that is connected to other portions of the barrel 12 by an adhesive, ultrasonic welding, or other suitable connecting processes, as are known in the art.

As previously described, the cap 24 is configured to be manually manipulated by the practitioner to move the cap 24 from the closed position to the open position. In order to allow for such manual manipulation, in some examples, the extension portion 80 comprises one or more slots 88 or openings extending through the sidewall 86 of the extension portion 80. The cap 24 can comprise beams or tabs 92 that extend through the slots 88 of the extension portion 80 for rotating the cap 24 relative to the syringe barrel 12. The cap 24 can further comprise a rotation ring 90 connected to the beams or tabs 92, which encircles the shield 74 of the cap 24. For example, as shown in FIGS. 4A and 4B, the cap 24 includes two tabs 92 or beams extending from opposite sides of the shield 74. The rotation ring 90 is attached to the tabs 92 and encircles the shield 74. The practitioner can grasp any portion of the rotation ring 90 when manipulating the cap 24 to move the cap 24 from the closed position to the open position. In some examples, the rotation ring 90 can include a grip portion, such as ridges 94 to make the ring 90 easier to grasp and manipulate. In particular, the rotation ring 90 can be configured so that the practitioner can grasp the ring 90 between a thumb and forefinger for rotating the cap 24. As previously described, rotating the cap 24 causes the tabs 92 or beams to slide through the slots 88, which causes the cap 24 to rotate relative to the barrel 12. Further, rotation of the cap 24 actuates the plug 63, causing the plug 63 to move away from the hole 56 so that fluid can flow from the barrel 12 of the syringe 10 through the cap 24.

In some examples, the slots 88 can include an axial portion or segment extending axially through the sidewall 86 from the distal end 84 of the extension portion 80. The axial portion or segment allows the cap 24 to be inserted into the extension portion 80 with the tabs 92 or beams protruding through the slot 88. The slot 88 also includes a circumferential portion or segment for rotating the cap 24 relative to the barrel 12 between the first position and the second position.

With reference again to FIGS. 2A-2C, in some examples, the slots 88 of the barrel 12 can include bumps 77 or protrusions protruding from an otherwise substantially flat surface of the slots 88. The bumps 77 can be positioned in the slots 88 to restrict or block the tabs 92 or beams from sliding easily through the slots 88. In some examples, the bumps 77 can be positioned to effectively lock the cap 24 in the extension portion 80 preventing the tabs 92 or beams from sliding through the axial segments of the slots 88 in order to remove the cap 24 from the extension portion 80.

With specific reference to FIG. 2C, the inner surface of the extension portion 80 can include structures for securing the barrel seal 54 in place seated at the proximal end of the extension portion 80. For example, as shown in FIG. 2C, the extension portion 80 can comprise protrusions, such as axially extending ridges 96, ribs, columns, or similar structures, on the inner surface of the extension portion 80 for restricting rotation of a barrel seal 54 positioned over the distal opening 18 of the barrel 12. As show in FIGS. 3A and 3B, the barrel seal 54 can include cut-outs 55, slots, or grooves positioned to receive the ridges 96 on the inner surface of the extension portion 80. The engagement between the ridges 96 and the cut-outs 55 of the barrel seal 54 maintains positioning of the barrel seal 54 and, in particular, prevents the barrel seal 54 from rotating as the cap 24 rotates between the closed position and the open position.

As shown in FIG. 2C, the extension portion 80 can also include a raised circumferential protrusion, such as a protruding ridge or locking ring 98. The locking ring 98 can be positioned to ensure that the barrel seal 54 remains seated against the distal end 16 of the barrel 12, preventing the barrel seal 54 from lifting away from the distal opening 18 of the barrel 12 as the cap 24 rotates between the first position and the second position. As previously described, the distal end 16 of the barrel 12 can also include the annular groove 62 (shown in FIG. 2C), which receives the ridge 58 or O-ring extending from the proximal surface of the barrel seal 54. The ridge 58 or O-ring can also contribute to maintaining positioning of the barrel seal 54 within the extension portion 80 of the barrel 12 by, for example, preventing the barrel seal 54 from sliding relative to the extension portion 80 or distal end 16 of the barrel 12. The ridge 58 or O-ring also effectively seals the distal opening 18 of the barrel 12 preventing fluid from collecting between the barrel seal 54 and the distal end 16 of the barrel 12.

Method of expelling fluid from a syringe with an integrated cap

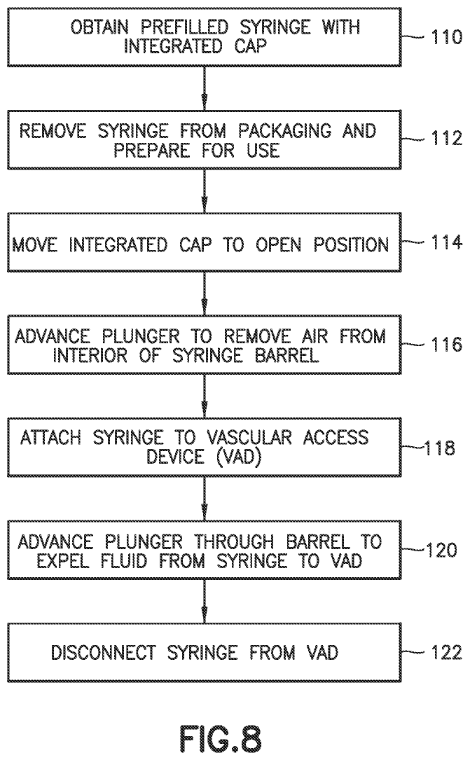

A flow chart illustrating steps for using the syringe 10 to expel fluid to a VAD is shown in FIG. 8. As shown in FIG. 8, at step 110, a practitioner initially obtains a prefilled syringe 10, in which the interior of the syringe barrel 12 is filled with a predetermined volume of the medical fluid F. For example, the prefilled syringe 10 can contain about 1 mL to about 50 mL or, preferably, about 5 mL to 20 mL of the medical fluid F. The cap 24 of the prefilled syringe 10 is initially in the closed position to ensure that the medical fluid F remains in the interior of the barrel 12 preventing contamination of the fluid and/or fluid leaks. In this initial or prefilled position, the stopper 38 is positioned proximate to the proximal end 14 of the barrel 12 and the plunger rod 40 extends proximally from the stopper 38 and through the proximal end 14 of the barrel 12.

At step 112, the practitioner prepares the syringe 10 for use by, for example, removing any packaging from the syringe 10 and removing a plunger cap, if present, that holds the plunger rod 40 in place. In the initial position, the cap 24 is in the closed position meaning that the fluid cannot pass through the connector 26. Instead, as shown by the arrows A4 in FIG. 6B, the fluid F is blocked from passing into the flow channel 28 of the connector 26 by the plug 63, which covers the hole 56 of the barrel seal 54.

At step 114, when ready to perform a flush or injection procedure, the practitioner moves the cap 24 to the open position. For example, the practitioner may grasp the rotation ring 90 of the cap 24 between a thumb and forefinger and rotate the ring 90 (as shown by arrow A1 in FIG. 1A), which causes the beams or tabs 92 of the cap 24 to move through the slots 88 of the extension portion 80. As the beams or tabs 92 slide through the slots 88, the cap 24 rotates from the closed position to the open position. As previously described, moving the cap 24 to the open position causes the plug 63 to rotate about the post 70 and move away from the hole 56, as shown by arrow A5 in FIGS. 6C and 7C. Movement of the plug 63 away from the hole 56 of the barrel seal 54 establishes fluid communication between the interior of the syringe barrel 12 and the flow passage or flow channel 28 of the connector 26 so that the medical fluid F can be expelled from the syringe 10.

At step 116, the practitioner next advances the stopper 38 through the barrel 12 in a distal direction (shown by arrow D in FIG. 1) by a small amount by, for example, pressing on the thumb plate 52 of the plunger rod 40, while grasping the finger flange 36 of the barrel 12 between, for example, a ring finger and an index fingers. This small distal movement of the stopper 38 removes air from the interior of the syringe barrel 12. At step 118, once any air is removed from the interior of the syringe barrel 12, the practitioner next attaches the connector 26 of the cap 24 to the VAD. For example, the practitioner may insert the distal end or distal tip 32 of the connector 26 into a corresponding port or valve of the VAD, in particular into a port or valve of a female needleless connector. Along with moving the syringe 10 distally to press the connector 26 into the septum of the female connector, the practitioner may also rotate the syringe 10 and integrated cap 24 in a direction shown by arrow A2 (shown in FIG. 1A) so that the threads 78 on the inner surface of the shield 74 engage corresponding threads on the female connector to secure the syringe 10 and integrated cap 24 to the female connector. As previously described, the direction of rotation for securing the shield 74 of the cap 24 to the female connector (shown by arrow A2 in FIG. 1A) may be opposite the direction of rotation to move the cap 24 from the closed position to the open position (shown by arrow A1 in FIG. 1A).

At step 120, with the cap 24 in the open position and attached to the VAD, the practitioner can move the stopper 38 in the distal direction (shown by arrow D in FIG. 1A) through the interior of the syringe barrel 12 using the plunger rod 40 in order to expel the medical fluid F from the syringe barrel 12 and through the flow passage or flow channel 28 of the connector 26. For example, as previously described, the practitioner may press the thumb press 52 of the plunger rod 40 with the thumb while grasping the finger flange 36 of the syringe barrel 12 to move the plunger rod 40 in the distal direction (shown by arrow D in FIG. 1A), which causes the stopper 38 to move distally through the syringe barrel 12 to force the medical fluid F from the syringe barrel 12, as shown by arrows A6 (shown in FIG. 7B). The practitioner continues to move the stopper 38 distally through the barrel 12 until all or substantially all of the medical fluid F is expelled from the barrel 12 through the flow passage or flow channel 28 of the connector 26 to the VAD.

At step 122, after the fluid F is expelled from the syringe barrel 12 through the connector 26, the syringe 10 can be disconnected from the VAD by rotating the syringe 10 in the direction A1 (shown in FIG. 1A) to release the threads 78 of the shield 74 from corresponding threads of the female connector.

As previously described, the prefilled syringe 10 of the present disclosure can be filled with a flush solution and used for performing a primary or pre-flush of the patient line of a VAD. In that case, after the flush solution is expelled from the syringe 10, the syringe 10 can be disconnected from the female connector and another syringe containing another medical fluid, such as a therapeutic agent, can be connected to the female connector for providing the therapeutic agent to the patient. In other examples, the prefilled syringe 10 of the present disclosure can contain a dose of the therapeutic agent to be delivered to the patient through the VAD. In that case, the syringe 10 can be attached to the female connector after flushing in order to provide the therapeutic agent to the patient. In other examples, the prefilled syringe 10 of the present disclosure can contain the flush solution and can be used for a post-flush action. For example, the prefilled syringe 10 can be connected to the VAD after the therapeutic agent is delivered to the patient through the VAD. The flush solution can be expelled from the prefilled syringe 10 through the connector 26 and the VAD to flush any remaining therapeutic agent in the VAD to the patient in order to ensure that a full dose of the therapeutic agent is provided to the patient. The post-flush procedure can also ensure that the patient line is clean by removing any debris or other contaminants from the patent line.

While examples of the syringe 10 comprising the integrated cap 24 and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A syringe comprising:
a barrel comprising a proximal end, a distal end comprising a distal opening, and a sidewall extending between the proximal end and the distal end;
a plunger positioned in the barrel for expelling fluid from the barrel through the distal opening; and
a cap connected to the distal end of the barrel comprising a connector defining a flow channel through the cap,
wherein the cap is movable relative to the barrel between a first position, where fluid flow through the distal opening of the barrel to the flow channel of the connector is prevented, and a second position, where fluid flow through the distal opening of the barrel to the flow channel of the connector can occur; and
a plug engaged to the cap, which moves as the cap moves from the first position to the second position, wherein, when the cap is in the first position, the plug is over top of the at least one hole of a barrel seal preventing fluid flow through the at least one hole of the barrel seal to the flow channel of the connector.

2. The syringe of claim 1, wherein the cap rotates about a longitudinal axis of the barrel to move from the first position to the second position.

3. The syringe of claim 1, further comprising a barrel seal positioned between the distal opening of the barrel and the cap, the barrel seal comprising at least one hole for fluid flow from the barrel to the flow channel of the connector, and
wherein the barrel seal comprises a disk portion positioned over the distal end of the barrel and an annular wall portion extending from the disk portion positioned against an inner surface of the barrel, wherein the at least one hole extends through the disk portion.

4. The syringe of claim 3, wherein the barrel seal comprises:
an annular ridge extending proximally from a proximal surface of the disk portion that is received within a corresponding annular groove on the distal end of the barrel, and
a lip extending radially inwardly from the annular wall portion of the barrel seal configured to engage the cap to retain a proximal portion of the cap within the barrel seal.

5. The syringe of claim 3,
wherein when the cap is in the second position, the plug is spaced apart from the at least one hole of the barrel seal to allow fluid flow from the barrel to the flow channel of the connector through the at least one hole of the barrel seal.

6. The syringe of claim 5, wherein the plug comprises a sealing portion that moves over the at least one hole of the barrel seal and at least one arm extending from the sealing portion, which is engaged to a portion of the cap.

7. The syringe of claim 5, wherein an end of the at least one arm of the plug is pivotally connected to an inner surface of the cap so that the plug can move to uncover the at least one hole of the barrel seal as the cap moves from the first position to the second position.

8. The syringe of claim 5, wherein the cap comprise at least one pair of protrusions extending radially inwardly from an inner surface of the cap, and wherein an end of the at least one arm of the plug is positioned between the pair of protrusions, such that the protrusions move the plug as the cap rotates from the first position to the second position.

9. The syringe of claim 5, wherein the plug comprises an elastomer seal configured to be positioned over the at least one hole of the barrel seal for sealing the at least one hole of the barrel seal when the cap is in the first position.

10. The syringe of claim 4, wherein the at least one hole of the barrel seal is axially aligned with the distal opening of the barrel, and wherein a diameter of the at least one hole of the barrel seal is smaller than a diameter of the distal opening of the barrel, and wherein the barrel seal remains stationary as the cap moves from the first position to the second position.

11. The syringe of claim 1, wherein the cap further comprises a shield extending about the connector, the shield comprising an inner surface comprising threads configured to engage threads of a female connector for securing the syringe and the cap to the female connector.

12. The syringe of claim 11, wherein the barrel further comprises an extension portion extending distally from the distal end of the barrel, and wherein the cap is connected to and at least partially enclosed by the extension portion, and
wherein the cap comprises at least one tab extending radially outward from the shield for rotating the cap relative to the barrel.

13. The syringe of claim 12, wherein the cap further comprises a rotation ring comprising the at least one tab, wherein the rotation ring extends about the shield of the cap and the extension portion of the barrel, and
wherein the rotation ring is configured to be rotated by a user to move the cap between the first position and the second position.

14. The syringe of claim 12, wherein the extension portion comprises at least one slot, and wherein the at least one tab of the cap extends through the slot so that the at least one tab can be grasped by a user to rotate the cap relative to the barrel.

15. The syringe of claim 14, wherein the at least one slot comprises at least one bump protruding from a surface of the slot, which restricts the at least one tab from sliding through the slot for locking the cap in the extension portion.

16. The syringe of claim 12, wherein the extension portion further comprises protrusions on an inner surface of the extension portion for restricting rotation of a barrel seal positioned over the distal opening of the barrel, and
wherein the protrusions comprise axially extending ridges, ribs, or columns on the inner surface of the extension portion.

17. A prefilled flushing syringe, comprising:
the syringe of claim 1; and
a predetermined volume of a fluid disposed in the barrel of the pre-filled syringe,
wherein the cap is initially in the first position, thereby preventing the fluid in the barrel from passing through the open distal end portion of the barrel to the flow channel of the connector.

18. The prefilled syringe of claim 17, wherein the fluid comprises a saline flush solution or a therapeutic agent.

19. A method for expulsion of fluid from the syringe of claim 1, the method comprising:
moving the cap from the first position to the second position, such that fluid flow from the barrel to the flow channel of the connector is permitted;
attaching the connector of the cap to a vascular access device; and
moving the plunger through the barrel to expel the fluid from the barrel and through the flow channel of the connector to the vascular access device.

20. The method of claim 19, wherein the cap is manually moved from the first position to the second position by grasping a portion of the cap with one hand and rotating the cap relative to a longitudinal axis of the syringe.
